(19) United States
(12) Patent Application Publication
(10) Pub. No.: US 2007/0119699 A1
(43) Pub. Date: May 31, 2007

(54) Apparatus and method for sanitizing air and spaces

(75) Inventors: William R. Chambers, Vienna, VA (US); Carlos Antonio Lima, Santiago (CL); Robert D. McDonald, Great Falls, VA (US); Terrance O. Woodbridge, Cary, NC (US)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: AirOCare, Inc., Rockville, MD (US)

(21) Appl. No.: 11/289,363

(22) Filed: Nov. 30, 2005

(51) Int. Cl. A61L 2/04; B01J 19/08

(52) U.S. Cl. 204/176; 422/186.07

(57) ABSTRACT

An apparatus for sanitizing air including a reaction unit for generating reactive oxygen species from oxygen in the air received in the reaction unit to be sanitized, wherein airborne contaminants in the air to be sanitized are neutralized by the generated concentration of reactive oxygen species before the air is discharged from the reaction unit. The reaction unit may further generate ozone from the oxygen in the air and the generated ozone is discharged with the air from the reaction unit to sanitize surfaces in the environment into which the sanitized air is discharged.

### FIELD OF THE INVENTION

The present invention relates to a method and apparatus for sanitizing air and spaces through the generation of reactive oxygen species.

### BACKGROUND OF THE INVENTION

Temperature changes and changes in the moisture in the air feeding into heating, ventilation, and air-conditioning (HVAC) systems increases the number micro-organisms in the air, producing increased colonies of certain fungi and bacteria, both of which are potentially harmful.

HVAC systems in office buildings, as well as hospitals, can be a source of various pathogens which spread infectious micro-organisms from one zone to another—a principal cause of Sick Building Syndrome, recognized by the World Health Organization as a threat to healthy work and living environments.

The purification of environments can be achieved through the use of ozone. Ozone has been used to purify air conditioning systems in buildings and to sanitize warehouses where products are stored. Despite its widespread use, this basic technique has the disadvantage of accumulating more ozone than is necessary in the treated environment, requiring the elimination of the excess ozone. Several different improvements in this method have been made in an attempt to control the levels of ozone in the environment being treated.

One such improvement provides high initial levels of ozone to the environment sufficient to produce the desired bacteriostatic or bacteriocidal effect. Later the levels of ozone are reduced so that they do not produce harmful effects to the products being treated or to humans in the environment.

However, the majority of the known systems for purifying closed areas with ozone are based on an ozone generator that utilizes a source of concentrated oxygen, for example bottled oxygen or a known pressurized oxygen generating system utilizing static discharge. When ozone is generated from a source of concentrated oxygen, the level of oxygen in the enclosure may rise along with the level of ozone. The increase in oxygen levels is due to the breakdown of ozone partially into new molecules of oxygen. An increase in the level of oxygen in enclosures containing natural perishable products enhances cellular metabolism and thus is detrimental to the storage of the perishable products.

One known method is applied to substantially closed rooms or rooms with a controlled atmosphere. The substantially closed room includes a closed circuit air conditioning system, such as a cooling system, for the preservation of perishable natural products. A known ozone generator is placed in proximity with the substantially closed room such that the ozone generator can draw in air from within the substantially closed room and liberate ozone into the substantially closed room. In contrast to other known ozonation systems, the known method utilizes oxygen from the air of the room in which the purification treatment is being applied to generate ozone. Because the method converts oxygen from the air into ozone, no increase in oxygen levels is observed in the closed room. Rather, the gaseous equilibrium is shifted so that there is maintenance of the level of oxygen in the enclosure.

The oxidative character of the ozone has a bacteriostatic and fungistatic effect in the short term, followed by a bacteriocidal and fungicidal effect. These effects combine with the lowered metabolism in a temperature cooled environment to reduce ripening, retard spoilage and thus preserve natural perishable products stored in the room.

However, the system does not provide an optimal means for efficiently sanitizing the air within the closed room.

### SUMMARY OF THE INVENTION

Therefore, it is an object of the present invention to provide an apparatus and method for generating reactive oxygen species and treating the air to be sanitized with the generated reactive oxygen species in order to efficiently sanitize air.

To attain the above object, according to a first aspect of the invention, there is provided an apparatus for sanitizing air including a reaction unit for generating reactive oxygen species from oxygen in air received in the reaction unit to be sanitized, wherein airborne contaminants in the received air are substantially neutralized by the generated reactive oxygen species before the air is discharged from the reaction unit. Preferably, the air to be sanitized comprises ambient air.

Preferably, the reactive oxygen species generated include at least one of singlet oxygen, atomic oxygen, superoxide, hydrogen peroxide, hydroxyl radical, and peroxynitrite.

The reaction unit preferably further generates ozone from the oxygen in the air and the generated ozone is discharged with the air from the reaction unit, wherein the ozone in the sanitized air acts as a sanitizing agent for sanitizing surfaces.

Preferably, the apparatus further includes an ultraviolet light source for illuminating the air discharged from the reaction unit with ultraviolet light to neutralize at least a portion of the ozone in the discharged air.

The ultraviolet light source is preferably selectively controlled to regulate the ozone discharged from the reaction unit, and the ultraviolet light has a frequency between about 280 nm and 290 nm. Preferably, the ultraviolet light has a frequency of 285 nm.

The apparatus preferably further includes an intake port for the receiving air to be sanitized, and an exhaust port for discharging the substantially sanitized air, wherein the reaction unit is disposed between the intake port and the exhaust port.

Preferably, the apparatus further includes a power supply capable of a high frequency and high voltage output electrically coupled with the reaction unit to create a corona discharge which splits the oxygen in the air into reactive oxygen species.

The power supply preferably includes an onboard intelligence for enabling the power supply to adjust to changing conditions within the reaction unit. Preferably, the onboard intelligence permits the power supply to adjust levels of reactive oxygen species generated by the reaction unit.

The apparatus preferably includes a plurality of sensors for measuring at least one of ozone levels, humidity, airflow, and temperature, and a programmable logic circuit for measuring performance of the apparatus based on data feedback from the plurality of sensors.

The apparatus preferably further includes a controller operably linked to the reaction unit and the programmable logic circuit, wherein the performance of the apparatus may be regulated based on the data feedback from the plurality of sensors.

Preferably, a plurality of reaction units fluidly linked in a serial manner, wherein the air to be sanitized passes through the plurality of reaction units in order maximize exposure of the air to be sanitized to the reactive oxygen species.

Preferably, the reaction unit includes at least one reaction chamber in which the reactive oxygen species are generated.

The reaction unit preferably includes a plurality of reaction chambers. The plurality of reaction chambers are preferably held in place within an array by a coupler arranged on the plurality of reaction chambers.

Preferably, the coupler includes a clamp for securing each of the plurality of reaction chambers in a desired location within the array and an electrically conductive contact cooperatively shaped with the clamp and electrically contacting each of the plurality of reaction chambers in the array.

Preferably, the contact is integrally formed with the clamp, or the contact is attached to the clamp by one of an adhesive and a mechanical fastener.

The reaction unit preferably includes a center support included in the array and secured by the clamp to provide additional structural integrity to the array.

Preferably, the coupler cooperates with an inner surface of a housing of the reaction unit to secure the reaction chambers within the housing,

The array is preferably fixed within the housing using electrically conductive contact studs that interact with the coupler, the contact studs securing the array in relation to the housing and electrically connecting with an electrically conductive contact, wherein the contact is electrically connected to each of the plurality of reaction chambers.

Preferably, an electrical connection between the reaction chamber of the reaction unit and the power supply is achieved through the contact studs.

Preferably. the reaction chamber comprises a glass tube lined with an inner stainless steel mesh and wrapped in an outer stainless steel mesh.

A second aspect of the invention provides a method for sanitizing air including generating reactive oxygen species from oxygen in ambient air, and sanitizing a flow of ambient air using the generated reactive oxygen species.

Preferably, the reactive oxygen species generated include at least one of singlet oxygen, atomic oxygen, superoxide, hydrogen peroxide, hydroxyl radical, and peroxynitrite. Preferably, the reactive oxygen species generated further include ozone.

A third aspect of the invention provides a method for sanitizing a room including generating reactive oxygen species comprising ozone and at least one of singlet oxygen, atomic oxygen, superoxide, hydrogen peroxide, hydroxyl radical, and peroxynitrite from oxygen in ambient air, sanitizing air with the generated reactive oxygen species, introducing the generated ozone into the sanitized air, and discharging the sanitized air with ozone into the room to be sanitized, wherein the ozone in the sanitized air acts as a sanitizing agent for sanitizing surfaces of the room.

Preferably, the method further includes selectively illuminating the discharged air with ultraviolet light to neutralize at least a portion of the ozone in the discharged air, wherein an amount of ozone in the discharged air can be controlled.

Preferably, the ultraviolet light has a frequency between about 280 nm and 290 nm.

Preferably, the ultraviolet light has a frequency of 285 nm.

These and other exemplary features and advantages of the present invention will become clear from the following description with reference to the accompanying drawings.

### BRIEF DESCRIPTION OF THE DRAWINGS

The foregoing and other exemplary purposes, aspects and advantages will be better understood from the following detailed description of an exemplary embodiment of the invention with reference to the drawings, in which:

FIG. 1 shows a block diagram of the apparatus of the exemplary embodiment of the invention.

FIG. 2 shows a perspective view of an exemplary embodiment of the reaction unit of the invention.

FIG. 3 shows an exploded perspective view of the reaction unit of FIG. 2.

FIG. 4 shows a further exploded perspective view of the reaction unit of FIG. 2.

FIG. 5 shows an exploded perspective view of the reaction chamber of FIG. 2.

FIG. 6 shows a perspective view of another embodiment of the reaction unit.

### DETAILED DESCRIPTION OF THE INVENTION

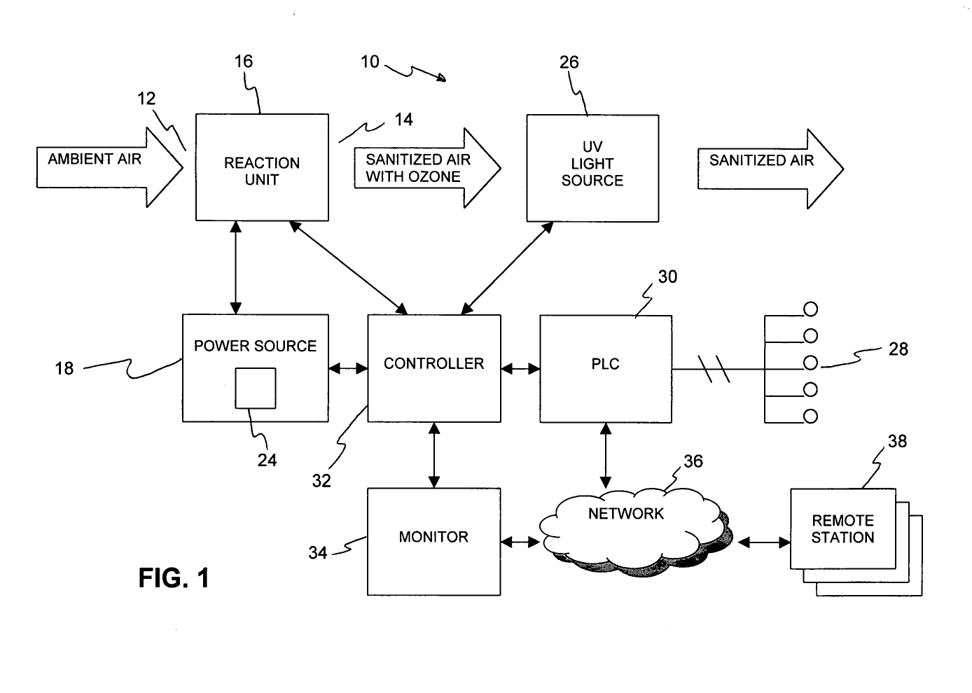

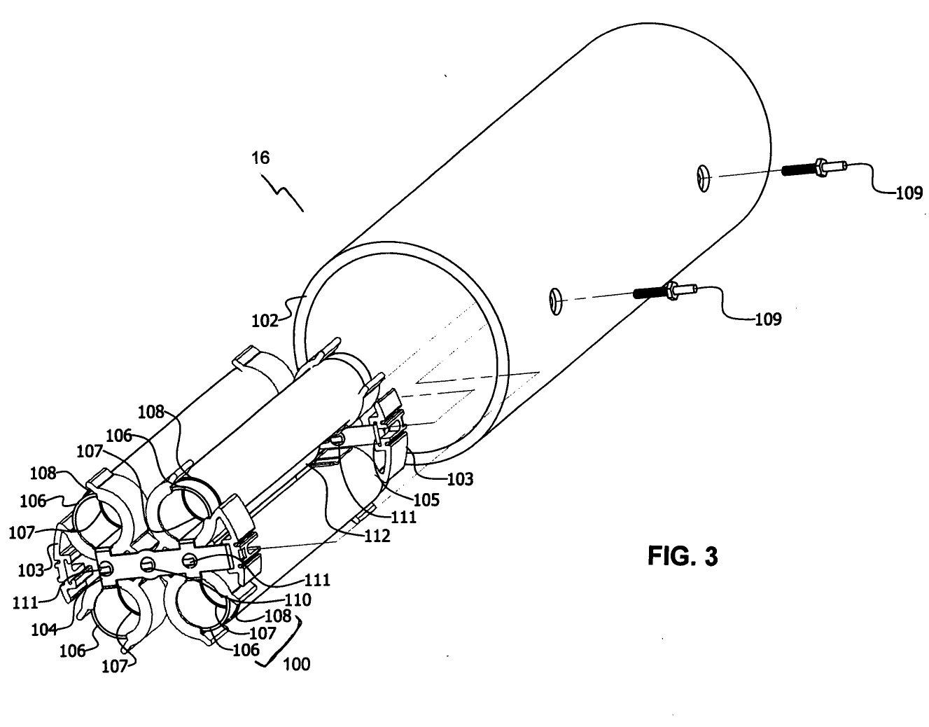

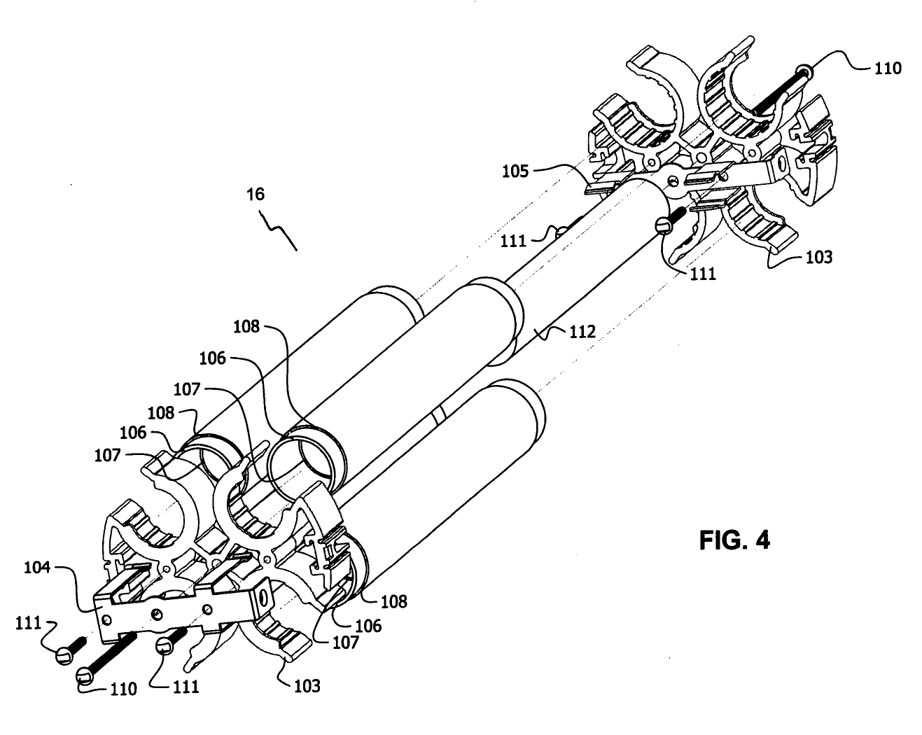

Referring now to the drawings, and more particularly to FIGS. 1-6, there are shown exemplary embodiments of the method and structures according to the present invention.

FIG. 1 shows an exemplary embodiment of the apparatus 10 for sanitizing air. The apparatus 10 includes an intake port 12 for receiving air to be sanitized and an exhaust port 14 for discharging substantially sanitized air. A reaction unit 16 is disposed between the intake port 12 and the exhaust port 14. The reaction unit 16 generates reactive oxygen species from oxygen (O₂) in the air received through the intake port 12.

The air received through the intake port 12 is preferably ambient air from the environment. The introduction of air into the reaction unit 16 may be mediated through a forced suction or by natural suction. When mediated through a forced suction, the apparatus 10 may contain a turbine which draws air into the reaction unit 16 through the intake port 12. Preferably, the air is drawn through a filter to remove dust and other macroscopic impurities that may be present in the air to be sanitized before the air enters the reaction unit 16.

The reaction unit 16 splits the oxygen in the air into large amounts of reactive oxygen species. The reactive oxygen species generated may include singlet oxygen (1O₂), ozone (O₃), atomic oxygen (O), superoxide (O₂—), hydrogen peroxide (H₂O₂), hydroxyl radical (OH—), and peroxynitrite (ONOO—). Even though many reactive oxygen species have a short half-life, they are effective sanitizing agents. Thus, as the air passes through the reaction unit 16, a large percentage of the airborne contaminants in the air received through the intake port 12 are neutralized by the generated reactive oxygen species before the air is exhausted through the exhaust port 14. In this manner, the reactive oxygen species generated in the reaction unit 16 act as a sanitizer of the air passing through the reaction unit 16.

One of the reactive oxygen species generated by the reaction unit 16 is ozone (O₃). The generated ozone is introduced into the air in the reaction unit 16, and the ozone also acts as a sanitizer of the air and environment. The ozone generated in the reaction unit 16 may be discharged with the air through the exhaust port 14. The ozone in the discharged air provides the beneficial preservative effects and acts as a sanitizer for any surfaces in the environment into which the air is discharged. Other reactive oxygen species, such as hydrogen peroxide, may also be discharged with the sanitized air and have sanitizing effects similar to ozone.

The apparatus may include a power supply 18 capable of producing high frequency and high voltage output. The power supply 18 is electrically coupled with the reaction unit 16 to create a corona discharge which splits the oxygen in the air into large amounts of reactive oxygen species. The power supply 18 provides power to the reaction unit 16.

The power supply 18 preferably includes an onboard intelligence 24 which enables the power supply 18 to adjust to changing conditions within the reaction unit 16. In this manner, the levels of reactive oxygen species generated within the reaction unit 16 can be maintained at desired levels regardless of changing conditions within the reactor unit 16. For example, the onboard intelligence 24 of the power supply 18 can compensate for variables that may affect the output of the reaction unit 16, such as changes in moisture content of the air to be sanitized or dust buildup within the reactor unit 16.

Further, the onboard intelligence 24 may allow for the dialing up and down of the levels of reactive oxygen species generated by the reaction unit 16. Preferably, the amount of reactive oxygen species generated by the reaction unit 16 is adjustable while maintaining continuous power to the reaction unit 16. However, one skilled in the art will recognize that the desired levels of reactive oxygen species may also be obtained by turning the reaction unit 16 on and off periodically.

The apparatus 10 may further include a source of ultraviolet (UV) light source 26 for illuminating the sanitized air discharged from the reaction unit 16 with UV light. By illuminating the discharged air with specific frequencies of UV light, it is possible to neutralize the ozone in the discharged sanitized air. In particular, UVB light having a frequency between about 280 nm and 290 nm will effectively neutralize the ozone. Preferably, the UV light source 26 emits UVB light having a frequency of 285 nm to achieve optimal neutralization of the ozone. In this manner, the UV light source 26 can turned on and off as necessary to regulate the ozone levels in the air ultimately discharged into the environment while maintaining high reactive oxygen species levels within the reaction unit 16 to permit continued sanitization of the air.

Thus, by placing the UV light source 26 downstream from the exhaust port 14, the air may continue to be sanitized by the reactive oxygen species generated in the reaction unit 16, while the ozone levels of the discharged air can be selectively controlled by using the UV light source 26 to neutralize the ozone in the discharged air. The apparatus 10 may further include an adjustable arm which can move the UV light source 26 so that it can be positioned for maximum effectiveness. The UV light source 26 may be configured using reflective surfaces in the form of a mirrored center array with concave light areas so that the UV light can be dispersed in a desired fashion, for example through the entire width of a duct, in order to maximize the ozone neutralization capability of the apparatus 10.

The apparatus 10 may further include other means for neutralizing the generated ozone. For example, the apparatus may include a heat source or carbon filtration means to neutralize the ozone in the discharged air. Additionally, the apparatus 10 may include means for neutralizing any other generated reactive oxygen species that may be discharged with the sanitized air from the reaction unit 16.

The apparatus 10 may further include a plurality of sensors and modules 28 located within the apparatus 10 and throughout the environment into which the sanitized air is discharged. The sensors and modules 28 are used to measure pertinent variables, such as ozone levels, humidity, airflow, and temperature of the air in and around the apparatus 10. A programmable logic circuit (PLC) 30 may be used to measure the performance of the apparatus 10 based on data feedback from the plurality of sensors and modules 28. The PLC 30 may store this information locally or report the information to a controller 32 which can be linked to the apparatus 10 and to a central monitor and monitoring system 34, such as a computer or other dedicated device.

In this manner, the PLC 30 may be used to monitor and control multiple functions of the apparatus 10 and facilitate data collection, retention, and reporting of performance (such as ozone output, etc.). The PLC 30 may also be used to monitor and control the power supply 16 through the onboard intelligence 24. Thus, the onboard intelligence 24 may use the feedback from the sensors and modules 28 to appropriately adjust the reaction unit 16 to provide the desired levels of reactive oxygen species.

The PLC 30 may be appropriately configured to make the information accessible to a remote computing device 38 over a network 36. The network 36 may include any known communications or networking means, for example, a Wide Area Network (WAN), Local Area Network (LAN), Internet, Bluetooth®, or any wireless connection. Thus, the PLC 30 may permit regulation and diagnosis of the apparatus 10 remotely by the computing device 38 over the networking means 36. It is to be understood that one or more of the onboard intelligence 24, PLC 30, controller 32, and monitoring system 34, or functions thereof, may be provided on a single appropriately configured computing device for monitoring and controlling the functions of the apparatus 10.

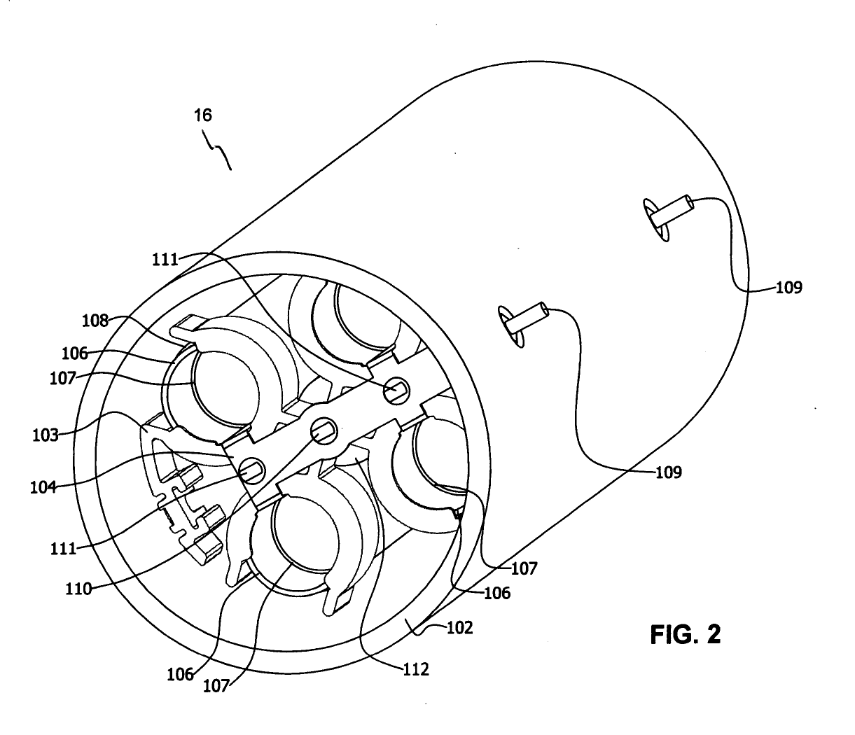

FIGS. 2-4 shows perspective and exploded views of an exemplary embodiment of the reaction unit 16 of the invention. The reaction unit 16 may consist of one or more reaction chambers 100 in which the reactive oxygen species are generated. The reaction chambers 100 may be arranged in an array within a housing 102. The housing 102 may consist of round polyvinyl chloride (PVC) pipe of appropriate size. However, it is understood that the housing may be of any desired shape or material. For example, the housing 102 may consist of the duct work of an HVAC system.

Preferably, the reaction chambers 100 are held in place within the array by a coupler arranged on both ends of the reaction chambers 100. The coupler may include a clamp 103 for securing the reaction chambers 100 in a desired location within the array. A center support rod 112 may be included in the array and appropriately secured by the clamp 103 to provide additional structural integrity to the array. The coupler may further include an electrically conductive contact 104,105 cooperatively shaped with the clamp 103 and contacting each of the reaction chambers 100 within the array. The contact 104 may be integrally formed with the clamp 103 or mechanically attached to the clamp 103 by an adhesive or mechanical fasteners 111.

The coupler preferably cooperates with an inner surface of the housing 102 to secure the reaction chambers 100 within the housing 102. The array may be fixed within the housing 102 using contact studs 109. The electrically conductive contact studs 109 pass through the housing 102 and interact with the coupler so as to fixedly secure the clamp 103 in relation to the housing 102 and electrically connect with the contacts 104,105. In this manner, the necessary electrical connections between the reaction chamber 100 of the reaction unit 16 and the power supply 18 may be achieved through the contact studs 109. However, one of ordinary skill in the art will recognize that the necessary electrical connections may be achieved by multiple means.

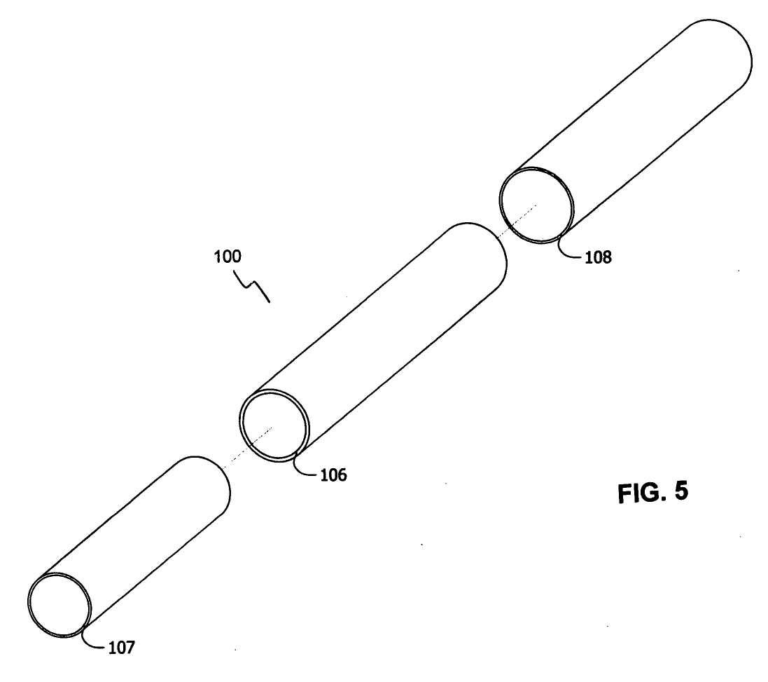

As shown in FIG. 5, the reaction chamber 100 may consist of a glass tube 106 lined with an inner stainless steel mesh 107 and wrapped in an outer stainless steel mesh 108. This configuration has been found to create a very effective corona that is able to generate a large amount of reactive oxygen species without using a static discharge and without producing material amounts of off gases, such as nitrous oxide. While a round configuration for the reaction chamber is shown, the reaction chambers for generating reactive oxygen species may include different configurations and materials. For example, the reaction chambers may be formed of a glass tube 106 wrapped in stainless steel mesh with a copper tube coated with gold inside the glass tube at specific gaps. The reaction chambers may also be formed using appropriately configured plates of glass, ceramic or other materials with metal mesh on opposite sides. The particular configuration may be chosen to comport with the desired application of the apparatus 10.

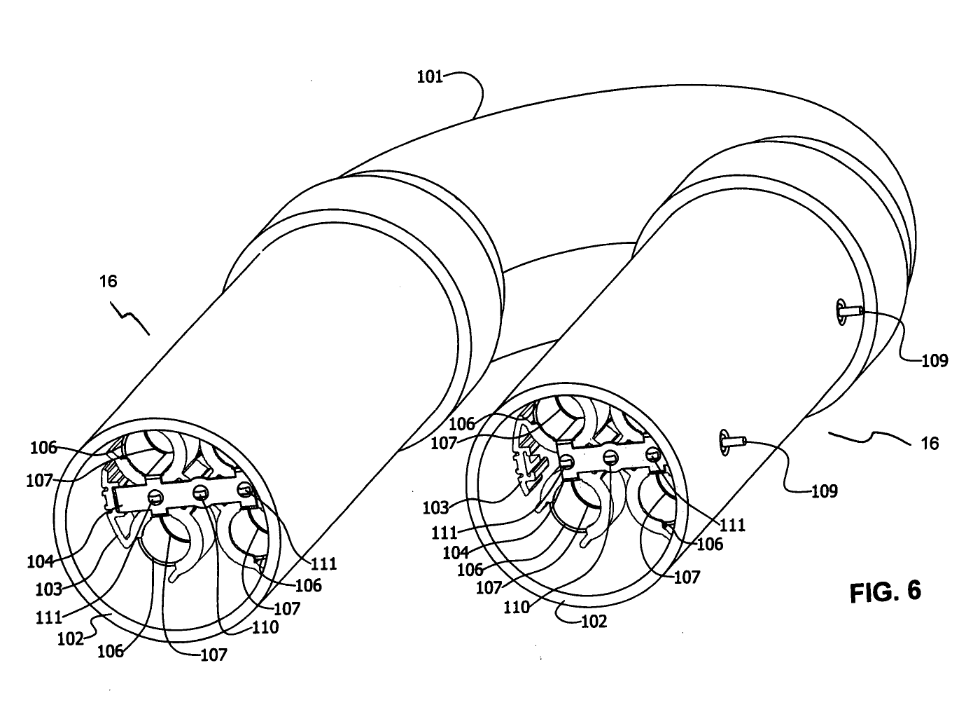

As shown in FIG. 6, the apparatus 10 may include a plurality of reaction units 16 fluidly linked in a serial manner. In this manner, the air to be sanitized may be passed through multiple reaction units 16 in order to maximize the exposure of air to the reactive oxygen species, therefore greatly increasing the effective sanitation of the air. While a U-shape is shown, it is understood that the reaction units 16 may be arranged in any manner depending on the space constraints of the desired application of the apparatus 10.

The reaction units 16 may be linked using an appropriate connector 101 that links the housings 102 of the reaction units 16. The reaction units 16 may be linked using a butt-plate. The butt-plate may include all the necessary electrical connections for the reaction units 16 to eliminate high-voltage wiring and avoid wiring problems. This also makes servicing the apparatus 10 more streamlined and efficient. The necessary electrical connections between the reaction units 16 may be achieved using military lock in rotation connectors connecting the butt-plate 24 and reaction units 16. Additionally, each reaction unit 16 may have its own power supply 18 in order to make the apparatus 10 highly scalable.

The apparatus 10 may be configured for general room sanitization applications where the apparatus 10, or components thereof, may be placed in the duct work of an HVAC system servicing the room to be sanitized. Alternatively, the apparatus 10 may be incorporated into the HVAC system of a facility to generally sanitize the air in the facility. Additionally, the apparatus 10 may be used to sanitize air to be introduced to a room from an outside source (make up air), as well as to treat exhaust air to remove smells and contaminants before releasing the air into the environment.

The apparatus 10 may be placed directly into a duct of an HVAC system so that some of the components are external to the duct in order to balance or reduce the weight of the apparatus 10 and create less stress on the duct work. For example, one or more reaction units 16 may be placed in the duct so that the air in the duct flows directly through the reaction unit 16 resulting in the generated reactive oxygen species sanitizing the air passing through, and the generated ozone cleaning the duct and being dispersed into the environment. As described above, a UV light source 26 may be placed downstream from the reaction units 16 in the duct to regulate the dispersion of ozone into the environment.

The level of ozone maintained in the environment into which the sanitized air containing ozone is dispersed, for example a room or building, may vary from as low as 0.02 PPM to higher levels depending on regulations and safe operating conditions based on human presence. One skilled in the art will realize that the optimum level will be determined based on the size, configuration, and contents of the room. Further, one skilled in the art will recognize that the levels of ozone maintained in the environment used by people may be limited by governmental regulation. For example, OSHA regulations stipulate that eight hours of exposure to 0.1 PPM ozone is acceptable and that fifteen minutes of exposure to 0.3 PPM ozone is acceptable. Use of higher concentrations may be dangerous. In the preferred embodiment, the level of ozone will be controlled and maintained, for example by the PLC 30, in accordance with governmental regulations. Higher levels of reactive oxygen species and ozone may be used during unoccupied periods for additional sanitation.

While the description refers to sanitizing air to be discharged into a room, space, or environment, it is to be understood that the invention can be applied to any defined environment. For example, an environment may be defined by solid surfaces or barriers, such as walls or product packaging, or defined by streams of forced gases, such as air screens or air curtains. Alternatively, the environment may be simply defined by the specific requirements of a desired application of the invention.

An exemplary application of the apparatus 10 would be for sanitizing sensitive areas of medical facilities, such as acute care areas and operating rooms. For example, the air circulation system of an operating room may include a network of ducts and vents that allow for the circulating of the air within the room without taking in air from outside the room. The apparatus 10, or elements thereof, may be placed in the duct work so that the air in the operating room may circulate through one or more reaction units 16. By including a UV light source 26, when the room is in use, the UV light source 26 may be turned on to prevent ozone from being dispersed in the room. But when the room is not occupied, the UV light source 26 may be turned off, allowing the generated ozone to circulate throughout the room and remove contaminants from surfaces inside the room. It is to be understood that the apparatus 10 may be employed in a wide variety of medical applications. For example, the sterilization of medical equipment storage cabinets and rooms, such as endoscope cabinets, and the sanitization of other rooms of medical facilities, such as waiting rooms, bathrooms, and food production areas.

In a similar manner, the apparatus 10 may be utilized in food processing environments to sanitize the air while food is being processed with workers present, provide the beneficial preservative effects of ozone while food is being stored (before and after processing), and sanitize the air and surfaces while the processing room is vacant. The apparatus 10 may also be configured into food processing equipment so that food is treated as it moves through the equipment, for example on a conveyor belt, automatic cutters and slicers and inspection areas. The product may be tumbled to promote uniform treatment. The apparatus 10 may also be configured to be placed in containers, trailers, and rail cars or as a component to a refrigeration system of such containers, trailers, and rail cars to sanitize the air therein while providing the beneficial preservative effects of ozone to any products stored therein.

Other exemplary applications of the apparatus include the provision or incorporation of the apparatus 10 into: grocery store display cases, such as deli counters and meat, fish and poultry display cases; floral display cases, both refrigerated and non-refrigerated; and HVAC systems of various public transportation means, such as cars, buses, trains, subways, or aircraft. The invention may be employed in pressurized environments, such as aircraft and positively or negatively pressurized rooms and structures. The apparatus 10 may also be incorporated into packing and production line equipment that blows air into bags as products are packed and sealed to sanitize the air blown into the bag and preserve the product therein, or into equipment that is integrated into a production line to sanitize the air and product before packaging.

As noted above, the apparatus 10 may also be incorporated into the HVAC system of public buildings in order to generally treat the air within the buildings. In this manner, the apparatus 10 may be used to sanitize the air and eliminate odors in the buildings. For example, office buildings, restaurants, malls, and the like would be particularly appropriate applications due to the large numbers of people that occupy the buildings and the need to sanitize the air in the buildings to provide a healthier, cleaner and more desirable environment for the occupants. The apparatus 10 may further be employed to sanitize air that is to be exhausted out of buildings in order to eliminate or reduce contaminants and odors emitted from the building into the surrounding environment.

In another exemplary application of the invention, the apparatus 10 may include sensors 28 for detecting potentially harmful agents in the environment. For example, the apparatus 10 may be incorporated into an HVAC system of a building and include appropriate sensors 28 for detecting noxious chemical or biological agents that may be unlawfully or accidentally released in or around the building. The apparatus 10 may be appropriately controlled to automatically operate in response to a positive detection of such agents by the sensor 28 in order to sanitize the air and protect the occupants of the building from the harmful agents.

In yet another application of the invention, the sanitized air discharged into the environment may be directed through a nozzle or jet to permit directional control of the sanitized air. In this manner, the sanitized air can be actively directed to a specific location or area requiring the sanitizing effect of the discharged air. Similarly, the invention may be incorporated into a means for creating air curtains or air doors. For example, an air curtain can be created to substantially enclose a specified space in order to contain and control any undesirable odors or emissions from contents within the created space, or, alternatively, sanitize or preserve the contents within the created space.

In a further exemplary application of the invention, the apparatus 10 may be incorporated into vacuum cleaner devices, for example stand-alone or centralized vacuum cleaners, wet-dry vacuums, and carpet cleaners, in order to sanitize air discharged from the cleaner. In this manner, any contaminants and odors inhaled by the cleaner would be sanitized and not discharged into the environment in which the cleaner was being utilized.

While the invention has been described in terms of several exemplary embodiments, those skilled in the art will recognize that the invention can be practiced with modification within the spirit and scope of the appended claims.

Further, it is noted that, Applicant's intent is to encompass equivalents of all claim elements, even if amended later during prosecution.

What is claimed is:

1. An apparatus for sanitizing air comprising:
a reaction unit for generating reactive oxygen species from oxygen in air received in the reaction unit to be sanitized,
wherein airborne contaminants in the received air are substantially neutralized by the generated reactive oxygen species before the air is discharged from the reaction unit.

2. The apparatus of claim 1, wherein the reactive oxygen species generated comprise at least one of singlet oxygen, atomic oxygen, superoxide, hydrogen peroxide, hydroxyl radical, and peroxynitrite.

3. The apparatus of claim 2, wherein the reaction unit further generates ozone from the oxygen in the air and the generated ozone is discharged with the air from the reaction unit, wherein the ozone in the sanitized air acts as a sanitizing agent for sanitizing surfaces.

4. The apparatus of claim 3, further comprising:
an ultraviolet light source for illuminating the air discharged from the reaction unit with ultraviolet light to neutralize at least a portion of the ozone in the discharged air.

5. The apparatus of claim 4, wherein the ultraviolet light source is selectively controlled to regulate the ozone discharged from the reaction unit.

6. The apparatus of claim 4, wherein the ultraviolet light has a frequency between about 280 nm and b 290 l nm.

7. The apparatus of claim 4, wherein the ultraviolet light has a frequency of 285 nm.

8. The apparatus of claim 1, further comprising:
an intake port for the receiving air to be sanitized; and
an exhaust port for discharging the substantially sanitized air,
wherein the reaction unit is disposed between the intake port and the exhaust port.

9. The apparatus of claim 1, further comprising:
a power supply capable of a high frequency and high voltage output electrically coupled with the reaction unit to create a corona discharge which splits the oxygen in the air into reactive oxygen species.

10. The apparatus of claim 9, wherein the power supply comprises an onboard intelligence for enabling the power supply to adjust to changing conditions within the reaction unit.

11. The apparatus of claim 12, wherein the onboard intelligence permits the power supply to adjust levels of reactive oxygen species generated by the reaction unit.

12. The apparatus of claim 1, further comprising:
a plurality of sensors for measuring at least one of ozone levels, humidity, airflow, and temperature; and
a programmable logic circuit for measuring performance of the apparatus based on data feedback from the plurality of sensality of sensors.

13. The apparatus of claim 12, further comprising:
a controller operably linked to the reaction unit and the programmable logic circuit, wherein the performance of the apparatus may be regulated based on the data feedback from the plurality of sensors.

14. The apparatus of claim 1, wherein the air to be sanitized comprises ambient air.

15. The apparatus of claim 1, further comprising:
a plurality of reaction units fluidly linked in a serial manner, wherein the air to be sanitized passes through the plurality of reaction units in order maximize exposure of the air to be sanitized to the reactive oxygen species.

16. The apparatus of claim 1, wherein the reaction unit comprises at least one reaction chamber in which the reactive oxygen species are generated.

17. The apparatus of claim 16, wherein the reaction unit comprises a plurality of reaction chambers.

18. The apparatus of claim 17, wherein the plurality of reaction chambers are held in place within an array by a coupler arranged on the plurality of reaction chambers.

19. The apparatus of claim 18, wherein the coupler comprises a clamp for securing each of the plurality of reaction chambers in a desired location within the array.

20. The apparatus of claim 19, wherein the reaction unit further comprises a center support included in the array and secured by the clamp to provide additional structural integrity to the array.

21. The apparatus of claim 19, wherein the coupler comprises an electrically conductive contact cooperatively shaped with the clamp and electrically contacting each of the plurality of reaction chambers in the array.

22. The apparatus of claim 21, wherein the contact is integrally formed with the clamp.

23. The apparatus of claim 21, wherein the contact is attached to the clamp by one of an adhesive and a mechanical fastener.

24. The apparatus of claim 18, wherein the coupler cooperates with an inner surface of a housing of the reaction unit to secure the reaction chambers within the housing.

25. The apparatus of claim 24, wherein the array is fixed within the housing using electrically conductive contact studs that interact with the coupler, the contact studs securing the array in relation to the housing and electrically connecting with an electrically conductive contact, wherein the contact is electrically connected to each of the plurality of reaction chambers.

26. The apparatus of claim 25, wherein an electrical connection between the reaction chamber of the reaction unit and the power supply is achieved through the contact studs.

27. The apparatus of claim 16, wherein the reaction chamber comprises a glass tube lined with an inner stainless steel mesh and wrapped in an outer stainless steel mesh.

28. A method for sanitizing air comprising:
generating reactive oxygen species from oxygen in ambient air; and
sanitizing a flow of ambient air using the generated reactive oxygen species.

29. The method of claim 28, wherein the reactive oxygen species generated comprise at least one of singlet oxygen, atomic oxygen, superoxide, hydrogen peroxide, hydroxyl radical, and peroxynitrite.

30. The method of claim 29, wherein the reactive oxygen species generated further comprise ozone.

31. A method for sanitizing a room, comprising:
generating reactive oxygen species comprising ozone and at least one of singlet oxygen, atomic oxygen, superoxide, hydrogen peroxide, hydroxyl radical, and peroxynitrite from oxygen in ambient air;
sanitizing air with the generated reactive oxygen species;
introducing the generated ozone into the sanitized air; and
discharging the sanitized air with ozone into the room to be sanitized, wherein the ozone in the sanitized air acts as a sanitizing agent for sanitizing surfaces of the room.

32. The method of claim 31, further comprising
selectively illuminating the discharged air with ultraviolet light to neutralize at least a portion of the ozone in the discharged air, wherein an amount of ozone in the discharged air can be controlled.

33. The method of claim 32, wherein the ultraviolet light has a frequency between about 280 nm and 290 nm.

34. The method of claim 32, wherein the ultraviolet light has a frequency of 285 nm.
